(19) United States
(12) Patent Application Publication
(10) Pub. No.: US 2021/0295952 A1
(43) Pub. Date: Sep. 23, 2021

(54) METHODS AND SYSTEMS FOR DETERMINING RESPONDERS TO TREATMENT

(71) Applicant: REGENERON PHARMACEUTICALS, INC., Tarrytown, NY (US)

(72) Inventors: Wen Zhang, Tarrytown, NY (US); Jing He, Tarrytown, NY (US)

(21) Appl. No.: 17/204,636

(22) Filed: Mar. 17, 2021

Related U.S. Application Data
(60) Provisional application No. 62/990,814, filed on Mar. 17, 2020.

(51) Int. Cl. G16B 40/00; G16B 5/20; G06N 20/00; G06N 5/04

(52) U.S. Cl. CPC G16B 40/00; G06N 5/04; G06N 20/00; G16B 5/20

(57) ABSTRACT

Methods, systems, and apparatuses for classifying a patient as a responder or a non-responder are described.

### CROSS-REFERENCE TO RELATED APPLICATIONS

This application claims the benefit of U.S. Provisional Patent Application No. 62/990,814, filed on Mar. 17, 2020, which is incorporated by reference herein in its entirety.

### BACKGROUND

One of the biggest issues facing the use of machine learning in is the lack of availability of large, annotated datasets. The annotation of data is not only expensive and time consuming but also highly dependent on the availability of expert observers. The limited amount of training data can inhibit the performance of supervised machine learning algorithms which often need very large quantities of data on which to train to avoid overfitting. So far, much effort has been directed at extracting as much information as possible from what data is available. One area in particular that suffers from lack of large, annotated datasets is analysis of biological data (e.g., clinical data), such as gene expression data. The ability to analyze gene data (e.g., gene expression data) to predict patient response to therapy is critical to patient care. However, in many instances, insufficient data are available to train machine learning algorithms to accurately predict patient response.

Thus, there is a need for improved systems and methods for determining and utilizing related gene data sets for use in machine learning applications. Therefore, it is an object of the invention to provide computer-implemented systems and methods that have improved capability to determine and utilize gene data sets for training machine learning applications to make predictions, including predicting patient response to therapy.

### SUMMARY

It is to be understood that both the following general description and the following detailed description are exemplary and explanatory only and are not restrictive.

In an embodiment, disclosed are methods comprising determining first gene data associated with a plurality of genes, determining second gene data associated with the plurality of genes, wherein the plurality of genes are sequenced from a plurality of tumor samples, wherein each tumor sample of the plurality of tumor samples is labeled as a responder or a non-responder, determining, based on the first gene data and the second gene data, a plurality of features for a predictive model, training, based on a first portion of the second gene data, the predictive model according to the plurality of features, testing, based on a second portion of the second gene data, the predictive model, and outputting, based on the testing, the predictive model.

In an embodiment, disclosed are methods comprising receiving baseline gene data associated with a plurality of genes for a subject, wherein the plurality of genes are sequenced from a tumor of the subject, providing, to a predictive model, the baseline gene data and determining, based on the predictive model, that the subject is a candidate for a therapeutic treatment.

In an embodiment, disclosed are methods comprising determining baseline gene expression data associated with a plurality of genes, wherein the plurality of genes are associated with a plurality of tumor samples, wherein each tumor sample of the plurality of tumor samples is labeled as a responder or a non-responder, determining, based on the plurality of genes, transcription regulator gene data, generating, based on the transcription regulator gene data and the plurality of genes, a transcription regulator (TR) network, determining, based on the TR network and the baseline gene expression data, an enrichment score associated with each transcription regulator gene of set of transcription regulator genes, and determining, based on the enrichment scores, one or more predictive transcription regulator genes of the set of transcription regulator genes.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of the present description serve to explain the principles of the methods and systems described herein:

FIG. 1 shows an example method;

FIG. 2 shows an example machine learning system;

FIG. 3 shows an example machine learning method;

FIG. 4 shows an example timeline for acquiring baseline and in-treatment gene expression data;

FIG. 5 shows normalized immune marker gene expression;

FIG. 6A shows differentially expressed genes determined by comparing the baseline gene expression data and the in-treatment gene expression for all patients (responder and non-responder);

FIG. 6B shows differentially expressed genes determined by comparing the baseline gene expression data and the in-treatment gene expression for responders only in pairs;

FIG. 6C shows differentially expressed genes determined by comparing the baseline gene expression data and the in-treatment gene expression for non-responders only in pairs;

FIG. 7 shows a heatmap on the right shows the top 50 differentially expressed genes of the overlapped differentially expressed genes from responders pairs only;

FIG. 8 shows differentially expressed genes between baseline responders and baseline non-responders;

FIG. 9 shows curated disease agnostic gene set data;

FIG. 10 shows predictive genes identified using the curated disease agnostic gene set data only;

FIG. 11 shows an example top performing gene signature;

FIG. 12 shows the performance of the example top performing gene signature;

FIG. 13 shows an example method for an example systems biology method for identifying predictive transcription regulator genes;

FIG. 14 shows the example predictive transcription regulator genes data identified from the systems biology method;

FIG. 15 shows a block diagram of an example computing device;

FIG. 16 shows an example method;

FIG. 17 shows an example method; and

FIG. 18 shows an example method.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. Similarly, when values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

Methods and systems are described for generating a machine learning classifier for treatment response prediction of a drug to a disease. Machine learning (ML) is a subfield of computer science that gives computers the ability to learn without being explicitly programmed. Machine learning platforms include, but are not limited to, naïve Bayes classifiers, support vector machines, decision trees, neural networks, and the like. In an embodiment, baseline (pre-treatment) gene expression data may be obtained for a plurality of patients prior to treatment and in-treatment gene expression data may be obtained for the plurality of patients during treatment. Patients that respond to the treatment and that did not respond to the treatment may be determined. In an embodiment, the baseline gene expression data and/or the in-treatment gene expression data may be analyzed to determine one or more predictive genes. The one or more predictive genes may predict a likelihood that a patient will be a responder or a non-responder to the drug. In an embodiment, the baseline gene expression data, the in-treatment gene expression data, and/or curated gene set enrichment data from one or more other studies may be analyzed to determine one or more predictive genes. In an embodiment, gene expression associated with one or more metabolic pathways may be analyzed to determine one or more predictive genes.

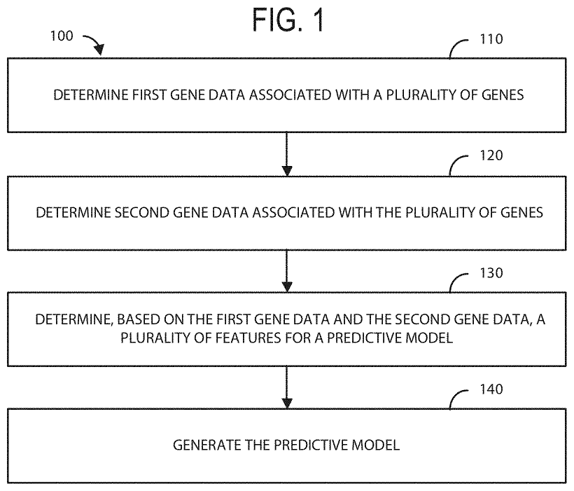

In an embodiment, (shown in FIG. 1) disclosed is a method 100 for generating a predictive model comprising determining first gene data associated with a plurality of genes at 110, determining second gene data associated with the plurality of genes at 120, determining, based on the first gene data and the second gene data, a plurality of features for a predictive model at 130, and generating, based on the plurality of features, the predictive model at 140.

The first gene data may comprise one or more of a list of the plurality of genes, sequence data associated with the list of genes, enrichment data, and/or the like. The plurality of genes in the first gene data may be associated with a first plurality of tumor samples. Each tumor sample of the first plurality of tumor samples may be labeled as a responder or a non-responder to a treatment.

The first gene data may be referred to as curated disease agnostic gene set data because the curated disease agnostic gene set data may be associated with the same treatment as the second gene data as described below, but may be associated with the same or a different disease. In an embodiment, the curated disease agnostic gene set data may not be associated with the same treatment or the same disease as the second gene data described below, but may be associated with one or more categories of gene sets, such as an immune cell type/function gene set, a tumor microenvironment component and signaling gene set, or a cancer cell proliferation and DNA repair gene set. The curated disease agnostic gene set data may contain at least one gene in common with the second gene data.

Determining the first gene data at 110 may comprise downloading/obtaining/receiving the curated disease agnostic gene set data may be obtained from various sources, including recent publications and/or publically available databases. The curated disease agnostic gene set data may comprise multiple gene data sets, associated with different conditions (e.g., melanoma, breast cancer, lung cancer, ovarian cancer, etc.) and may be generated from various data types and/or platforms (e.g., bulk RNA-seq, single cell RNA-seq, NanoString, etc.). The methods described herein may utilize the curated disease agnostic gene set data to improve identification of predictor genes.

The second gene data may comprise one or more of a list of the plurality of genes, sequence data associated with the list of genes, enrichment data, and/or the like. The plurality of genes in the second gene data may be sequenced from a second plurality of tumor samples. Each tumor sample of the second plurality of tumor samples may be labeled as a responder or a non-responder. Determining the second gene data associated with the plurality of genes at 120 may comprise determining baseline (pre-treatment) gene expression levels for each tumor associated with the second plurality of tumor samples. Each tumor may be treated with a therapeutic and, post-treatment, it may be determined which tumors are responders or non-responders to the therapeutic. The baseline (pre-treatment) gene expression levels for each tumor may then be labeled as responder or non-responder and stored as the second gene data. In an embodiment, the baseline gene expression data and the in-treatment gene expression data may comprise one or more of RNA-Seq data, TCR-seq data, DNA-seq data, and/or imaging data. RNA-Seq data may indicate the presence and quantity of RNA in a biological sample. TCR-seq data may indicate the presence and quantity of T-cell receptors in a biological sample. DNA-seq data may indicate the presence and quantity of DNA and/or a mutation in a biological sample.

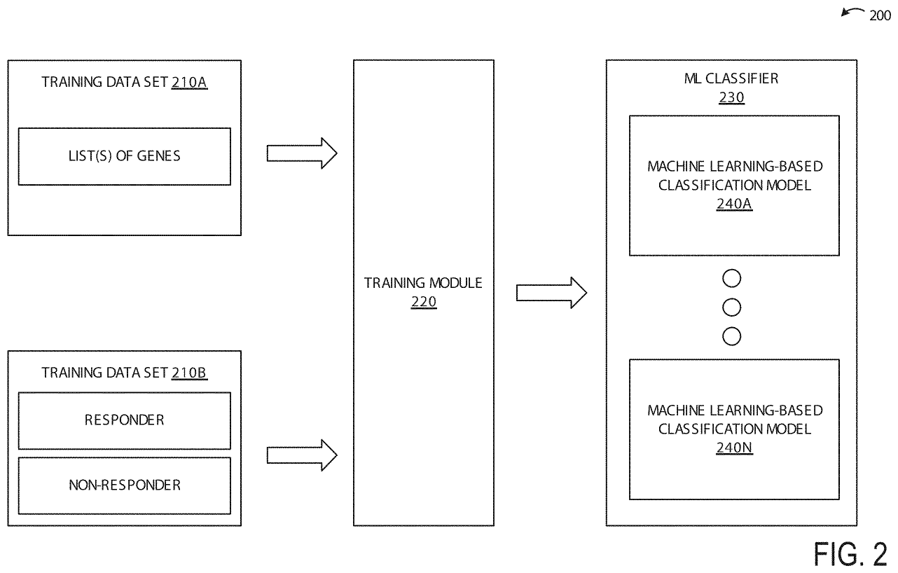

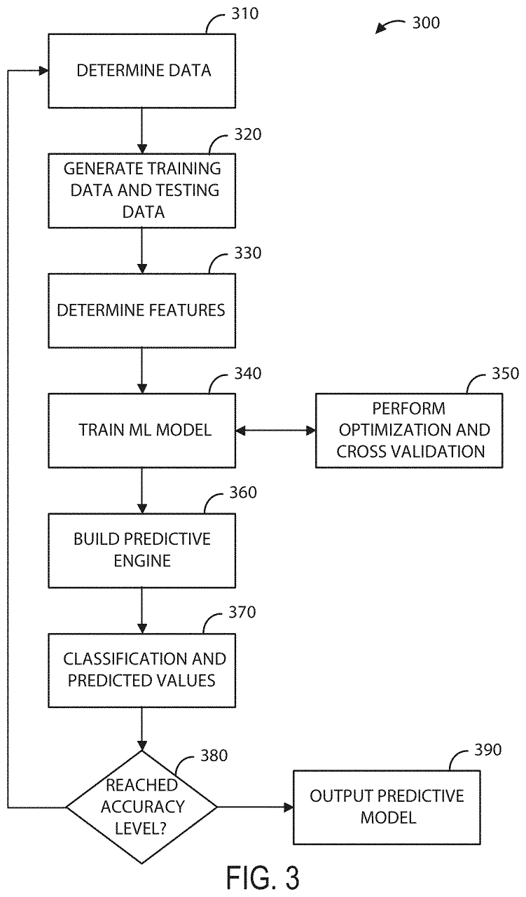

Determining, based on the first gene data and the second gene data, the plurality of features for a predictive model at 130 and generating, based on the plurality of features, the predictive model at 140 are described with regard to FIG. 2 and FIG. 3.

In an embodiment, a predictive model (e.g., a machine learning classifier) may be generated to classify a patient as a responder or a non-responder based on analyzing the patient's baseline gene expression data. The predictive model may be trained according to the first gene data (e.g., curated disease agnostic gene set data) and the second gene data (e.g., baseline gene expression data and/or in-treatment gene expression data). The baseline gene expression data and the in-treatment gene expression data may relate to a single study involving the same patient cohort treated with a drug/treatment. The curated disease agnostic gene set data may contain at least one gene in common with the baseline gene expression data and may relate to one or more different studies involving a different patient cohort(s) treated with the same or different drug/treatment and having the same or different disease. In an embodiment, one or more features of the predictive model may be extracted from one or more of the baseline gene expression data, the in-treatment gene expression data, and/or the curated disease agnostic gene set data. In an embodiment, one or more features of the predictive model may be extracted from a combination of one or more of a portion of the baseline gene expression data and/or a portion of the curated disease agnostic gene set data.

As shown in FIG. 2, a system 200 is described herein that is configured to use machine learning techniques to train, based on an analysis of one or more training data sets 210A-210B by a training module 220, at least one machine learning-based classifier 230 that is configured to classify baseline gene expression data as being associated with a responder or a non-responder. In an embodiment, the training data set 210A (e.g., the first gene data) may comprise the curated disease agnostic gene set data from one or more studies (e.g., one or more lists of genes). In an embodiment, the training data set 210A may comprise only curated disease agnostic gene set data or only a portion of the curated disease agnostic gene set data. In an embodiment, the training data set 210B (e.g., the second gene data) may comprise labeled baseline gene expression data. In an embodiment, the training data set 210B may comprise only the labeled baseline gene expression data or only a portion of the labeled baseline gene expression data. The labels may comprise responder and non-responder.

The second gene data for each patient may be randomly assigned to the training data set 210B or a testing data set. In some implementations, the assignment of data to a training data set or a testing data set may not be completely random. In this case, one or more criteria may be used during the assignment, such as ensuring that similar numbers of patients with different responder/non-responder statuses are in each of the training and testing data sets. In general, any suitable method may be used to assign the data to the training or testing data sets, while ensuring that the distributions of responder/non-responder statuses are somewhat similar in the training data set and the testing data set. In an embodiment, 75% of the labeled baseline gene expression data may be assigned to the training data set 210B and 25% of the labeled baseline gene expression data may be assigned to the test data set.

In an embodiment, the training module 220 may train the machine learning-based classifier 230 by extracting a feature set from the first gene data (e.g., the curated disease agnostic gene set data) in the training data set 210A according to one or more feature selection techniques. In an embodiment, the training module 220 may further define the feature set obtained from the training data set 210A by applying one or more feature selection techniques to the second gene data (e.g., the labeled baseline gene expression data) in the training data set 210B that includes statistically significant features of positive examples (e.g., responder) and statistically significant features of negative examples (e.g., non-responder).

In an embodiment, the training module 220 may extract a feature set from the training data set 210A and/or the training data set 210B in a variety of ways. The training module 220 may perform feature extraction multiple times, each time using a different feature-extraction technique. In an embodiment, the feature sets generated using the different techniques may each be used to generate different machine learning-based classification models 240. In an embodiment, the feature set with the highest quality metrics may be selected for use in training. The training module 220 may use the feature set(s) to build one or more machine learning-based classification models 240A-240N that are configured to indicate whether or not new data is associated with a responder or a non-responder.

In an embodiment, the training data set 210B may be analyzed to determine any dependencies, associations, and/or correlations between measured gene expression levels and the responder/non-responder statuses of the patients in the training data set 210B. The identified correlations may have the form of a list of genes that are differentially expressed for samples that are associated with different responder/non-responder statuses. In an embodiment, the training data set 210A may be analyzed to determine one or more lists of genes that have at least one gene in common with the training data set 210B. The genes may be considered as features (or variables) in the machine learning context. The term “feature,” as used herein, may refer to any characteristic of an item of data that may be used to determine whether the item of data falls within one or more specific categories. By way of example, the features described herein may comprise one or more genes.

In an embodiment, a feature selection technique may comprise one or more feature selection rules. The one or more feature selection rules may comprise a gene occurrence rule. The gene occurrence rule may comprise determining which genes in the training data set 210A occur over a threshold number of times and identifying those genes that satisfy the threshold as candidate features. For example, any genes that appear greater than or equal to 2 times in the training data set 210A may be considered as candidate features. Any genes appearing less than 2 times may be excluded from consideration as a feature.

In an embodiment, the one or more feature selection rules may comprise an expression level rule. The expression level rule may comprise determining which genes in the baseline gene expression data in the training data set 210B have expression levels that exceed an expression threshold and identifying those genes that satisfy the threshold as candidate features. For example, any genes that have expression levels that are greater than or equal to 2 Transcripts Per Million (TPM) may be considered as candidate features. Any genes that have expression levels less than 2 TPM may be excluded from consideration as a feature.

In an embodiment, the one or more feature selection rules may comprise a significance rule. The significance rule may comprise determining, from the baseline gene expression data in the training data set 210B, responder gene expression data and non-responder gene expression data. As the baseline gene expression data in the training data set 210B are labeled as responder or non-responder, the labels may be used to determine the responder gene expression data and non-responder gene expression data. The gene expression levels of the genes in the responder gene expression data may be compared to the gene expression levels of those same genes in the non-responder gene expression data. Genes having statistically significant (e.g., p-value) differential expression may be determined based on the comparison. For example, those genes with differential expression having a p-value less than a threshold may be selected as candidate features. The threshold may be, for example, 0.1. Those genes with differential expression having a p-value greater than or equal to the threshold may be excluded from consideration as a feature.

In an embodiment, the one or more feature selection rules may comprise a tumor mutational burden (TMB) rule. The TMB rule may comprise determining a TMB value for each gene contained in the training data set 210A and/or the training data set 210B. The value of TMB may be used as a feature.

In an embodiment, a single feature selection rule may be applied to select features or multiple feature selection rules may be applied to select features. In an embodiment, the feature selection rules may be applied in a cascading fashion, with the feature selection rules being applied in a specific order and applied to the results of the previous rule. For example, the gene occurrence rule may be applied to the training data set 210A to generate a first list of genes. The expression level rule may be applied to genes in the first list to determine which genes of the first list satisfy the expression level rule in the training data set 210B and to generate a second list of genes. The significance rule may be applied to genes in the second list of genes to determine which genes of the second list satisfy the significance rule in the training data set 210B and to generate final list of candidate genes (features).

The final list of candidate genes may be analyzed according to additional feature selection techniques to determine one or more candidate gene signatures (e.g., groups of genes that may be used to predict whether a patient is a responder or non-responder). Any suitable computational technique may be used to identify the candidate gene signatures using any feature selection technique such as filter, wrapper, and/or embedded methods. In an embodiment, one or more candidate gene signatures may be selected according to a filter method. Filter methods include, for example, Pearson's correlation, linear discriminant analysis, analysis of variance (ANOVA), chi-square, combinations thereof, and the like. The selection of features according to filter methods are independent of any machine learning algorithms. Instead, features may be selected on the basis of scores in various statistical tests for their correlation with the outcome variable (e.g., responder/non-responder).

In an embodiment, one or more candidate gene signatures may be selected according to a wrapper method. A wrapper method may be configured to use a subset of features and train a machine learning model using the subset of features. Based on the inferences that drawn from a previous model, features may be added and/or deleted from the subset. Wrapper methods include, for example, forward feature selection, backward feature elimination, recursive feature elimination, combinations thereof, and the like. In an embodiment, forward feature selection may be used to identify one or more candidate gene signatures. Forward feature selection is an iterative method that begins with no feature in the machine learning model. In each iteration, the feature which best improves the model is added until an addition of a new variable does not improve the performance of the machine learning model. In an embodiment, backward elimination may be used to identify one or more candidate gene signatures. Backward elimination is an iterative method that begins with all features in the machine learning model. In each iteration, the least significant feature is removed until no improvement is observed on removal of features. In an embodiment, recursive feature elimination may be used to identify one or more candidate gene signatures. Recursive feature elimination is a greedy optimization algorithm which aims to find the best performing feature subset. Recursive feature elimination repeatedly creates models and keeps aside the best or the worst performing feature at each iteration. Recursive feature elimination constructs the next model with the features remaining until all the features are exhausted. Recursive feature elimination then ranks the features based on the order of their elimination.

In an embodiment, one or more candidate gene signatures may be selected according to an embedded method. Embedded methods combine the qualities of filter and wrapper methods. Embedded methods include, for example, Least Absolute Shrinkage and Selection Operator (LASSO) and ridge regression which implement penalization functions to reduce overfitting. For example, LASSO regression performs L1 regularization which adds a penalty equivalent to absolute value of the magnitude of coefficients and ridge regression performs L2 regularization which adds a penalty equivalent to square of the magnitude of coefficients.

After training module 220 has generated a feature set(s), the training module 220 may generate a machine learning-based classification model 240 based on the feature set(s). Machine learning-based classification model, may refer to a complex mathematical model for data classification that is generated using machine-learning techniques. In one example, this machine learning-based classifier may include a map of support vectors that represent boundary features. By way of example, boundary features may be selected from, and/or represent the highest-ranked features in, a feature set.

In an embodiment, the training module 220 may use the feature sets extracted from the training data set 210A and/or the training data set 210B to build a machine learning-based classification model 240A-240N for each classification category (e.g., responder, non-responder). In some examples, the machine learning-based classification models 240A-240N may be combined into a single machine learning-based classification model 240. Similarly, the machine learning-based classifier 230 may represent a single classifier containing a single or a plurality of machine learning-based classification models 240 and/or multiple classifiers containing a single or a plurality of machine learning-based classification models 240.

The extracted features (e.g., one or more candidate genes and/or candidate gene signatures derived from the final list of candidate genes) may be combined in a classification model trained using a machine learning approach such as discriminant analysis; decision tree; a nearest neighbor (NN) algorithm (e.g., k-NN models, replicator NN models, etc.); statistical algorithm (e.g., Bayesian networks, etc.); clustering algorithm (e.g., k-means, mean-shift, etc.); neural networks (e.g., reservoir networks, artificial neural networks, etc.); support vector machines (SVMs); logistic regression algorithms; linear regression algorithms; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like. The resulting machine learning-based classifier 230 may comprise a decision rule or a mapping that uses the expression levels of the genes in the candidate gene signature to assign a patient to a class (responder/non-responder).

The candidate gene signature and the machine learning-based classifier 230 may be used to predict the responder/non-responder statuses of the test samples in the testing data set. In one example, the result for each test sample includes a confidence level that corresponds to a likelihood or a probability that the corresponding test sample belongs in the predicted responder/non-responder status. The confidence level may be a value between zero and one, that represents a likelihood that the corresponding test sample belongs to a responder/non-responder status. In one example, when there are two statuses (e.g., responder and non-responder), the confidence level may correspond to a value p, which refers to a likelihood that a particular test sample belongs to the first status. In this case, the value 1−p may refer to a likelihood that the particular test sample belongs to the second status. In general, multiple confidence levels may be provided for each test sample and for each candidate gene signature when there are more than two statuses. A top performing candidate gene signature may be determined by comparing the result obtained for each test sample with the known responder/non-responder status for each test sample. In general, the top performing candidate gene signature will have results that closely match the known responder/non-responder statuses.

The top performing candidate gene signature may be used to predict the responder/non-responder status of an individual. For example, baseline gene expression data for a potential patient may be determined/received. The baseline gene expression data for the potential patient may be provided to the machine learning-based classifier 230 which may, based on the top performing candidate gene signature, classify the potential patient as a responder or as a non-responder. If classified as a responder, the potential patient may be treated with the drug/treatment. If classified as a non-responder, an alternate treatment may be provided to the potential patient.

FIG. 3 is a flowchart illustrating an example training method 300 for generating the machine learning-based classifier 230 using the training module 220. The training module 220 can implement supervised, unsupervised, and/or semi-supervised (e.g., reinforcement based) machine learning-based classification models 240. The method 300 illustrated in FIG. 3 is an example of a supervised learning method; variations of this example of training method are discussed below, however, other training methods can be analogously implemented to train unsupervised and/or semi-supervised machine learning models.

The training method 300 may determine (e.g., access, receive, retrieve, etc.) first gene data (e.g., lists of genes, expression data, etc. . .) of one or more populations of patients and second gene data of one or more other populations of patients at 310. The first gene data may contain one or more datasets, each dataset associated with a particular study. Each study may include one or more genes in common with the second gene data. Each study may or may not involve the same drug/treatment and may or may not be associated with the same, or different, disease/condition. Each study may involve different patient populations, although it is contemplated that some patient overlap may occur. In an embodiment, each dataset may include a list of differentially expressed genes. The second gene data may contain may contain one or more datasets, each dataset associated with a particular study, different from those of the first gene data set. Each study may include one or more genes in common with the first gene data. Each study may or may not involve the same drug/treatment and may or may not be associated with the same, or different, disease/condition. Each study may involve different patient populations, although it is contemplated that some patient overlap may occur. In an embodiment, each dataset may include a labeled list of differentially expressed genes. In another embodiment, each dataset may comprise labeled baseline gene expression data. In another embodiment, each dataset may further include labeled in-treatment gene expression data. The labels may comprise responder or non-responder. The gene expression data may comprise whole exome sequencing data, whole genome sequencing data, RNA-seq data, combinations thereof, and the like. The gene expression data may comprise an identification of genes present in a biological sample of a patient and at what expression level. For example, in the case of RNA-seq data, the quantity and sequences of RNA in a biological sample may be determined using next generation sequencing (NGS).

The training method 300 may generate, at 320, a training data set and a testing data set. The training data set and the testing data set may be generated by randomly assigning labeled gene expression data of individual patients from the second gene data to either the training data set or the testing data set. In some implementations, the assignment of patients as training or test samples may not be completely random. In an embodiment, only the labeled baseline gene expression data for a specific study may be used to generate the training data set and the testing data set. In an embodiment, a majority of the labeled baseline gene expression data for the specific study may be used to generate the training data set. For example, 75% of the labeled baseline gene expression data for the specific study may be used to generate the training data set and 25% may be used to generate the testing data set. In another embodiment, only the labeled in-treatment gene expression data for the specific study may be used to generate the training data set and the testing data set.

The training method 300 may determine (e.g., extract, select, etc.), at 330, one or more features that can be used by, for example, a classifier to differentiate among different classifications (e.g., responder vs. non-responder). The one or more features may comprise a set of genes. In an embodiment, the training method 300 may determine a set features from the first gene data. In another embodiment, the training method 300 may determine a set of features from the second gene data. In another embodiment, a set of features may be determined from gene data from a study different than the study associated with the labeled gene data of the training data set and the testing data set. In other words, gene data from the different study (e.g., the curated disease agnostic gene data) may be used for feature determination, rather than for training a machine learning model. In an embodiment, the training data set may be used in conjunction with the gene data from the different study to determine the one or more features. The gene data from the different study may be used to determine an initial set of features, which may be further reduced using the training data set.

The training method 300 may train one or more machine learning models using the one or more features at 340. In one embodiment, the machine learning models may be trained using supervised learning. In another embodiment, other machine learning techniques may be employed, including unsupervised learning and semi-supervised. The machine learning models trained at 340 may be selected based on different criteria depending on the problem to be solved and/or data available in the training data set. For example, machine learning classifiers can suffer from different degrees of bias. Accordingly, more than one machine learning models can be trained at 340, optimized, improved, and cross-validated at 350.

The training method 300 may select one or more machine learning models to build a predictive model at 360 (e.g., a machine learning classifier). The predictive model may be evaluated using the testing data set. The predictive model may analyze the testing data set and generate classification values and/or predicted values at 370. Classification and/or prediction values may be evaluated at 380 to determine whether such values have achieved a desired accuracy level. Performance of the predictive model may be evaluated in a number of ways based on a number of true positives, false positives, true negatives, and/or false negatives classifications of the plurality of data points indicated by the predictive model. For example, the false positives of the predictive model may refer to a number of times the predictive model incorrectly classified a patient as a responder that was in reality a non-responder. Conversely, the false negatives of the predictive model may refer to a number of times the machine learning model classified one or more patients as a non-responder when, in fact, the patient was a responder. True negatives and true positives may refer to a number of times the predictive model correctly classified one or more patients as a responder or a non-responder. Related to these measurements are the concepts of recall and precision. Generally, recall refers to a ratio of true positives to a sum of true positives and false negatives, which quantifies a sensitivity of the predictive model. Similarly, precision refers to a ratio of true positives a sum of true and false positives.

When such a desired accuracy level is reached, the training phase ends and the predictive model may be output at 390; when the desired accuracy level is not reached, however, then a subsequent iteration of the training method 300 may be performed starting at 310 with variations such as, for example, considering a larger collection of gene expression data.

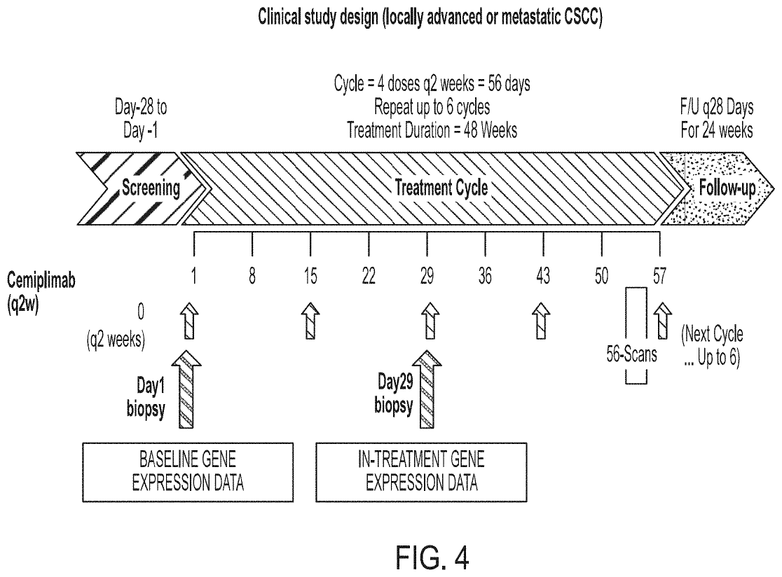

FIG. 4 shows gene expression data (e.g., RNA-seq data) acquired from a cohort of patients who were treated with a drug for a disease (the CSCC data). The cohort of patients were treated with Cemiplimab over a 48 week period for treatment of cutaneous squamous cell cancer (CSCC). All patients in the cohort underwent baseline, pre-treatment screening before starting treatment. During baseline (pre-treatment) screening, a biopsy sample of each patient's tumor was obtained and each biopsy sample sequenced using Next-Generation Sequencing (NGS) techniques. Baseline gene expression data for each patient was thus obtained prior to treatment (e.g., Day 1). After treatment began, another biopsy sample of each patient's tumor was obtained and each biopsy sample sequenced using NGS techniques to obtain in-treatment gene expression data. In-treatment gene expression data for each patient was thus obtained during the treatment period (e.g., Day 29). While described as being determined in the context of Cemiplimab and CSCC, it is to be understood that the methods and systems described herein may be applied to any treatment and for any condition. Accordingly, baseline gene expression data and in-treatment gene expression data may be determined for any drug/treatment and for any disease/condition. The baseline gene expression data and the in-treatment gene expression data may comprise one or more of RNA-Seq data, TCR-seq data, DNA-seq data, and/or imaging data.

After treatment, the patients were classified as responders or non-responders. Patients may be counted as responders if they displayed greater than 30% decrease in tumor volume. Other techniques may be used to classify patients as responders or non-responders, including varying the percent decrease in tumor volume (e.g., 10%, 20%, 40%, 50%, 60%, 70%, 80%, 100%). The baseline gene expression data and the in-treatment gene expression data for each patient were then be labeled as responder or non-responder.

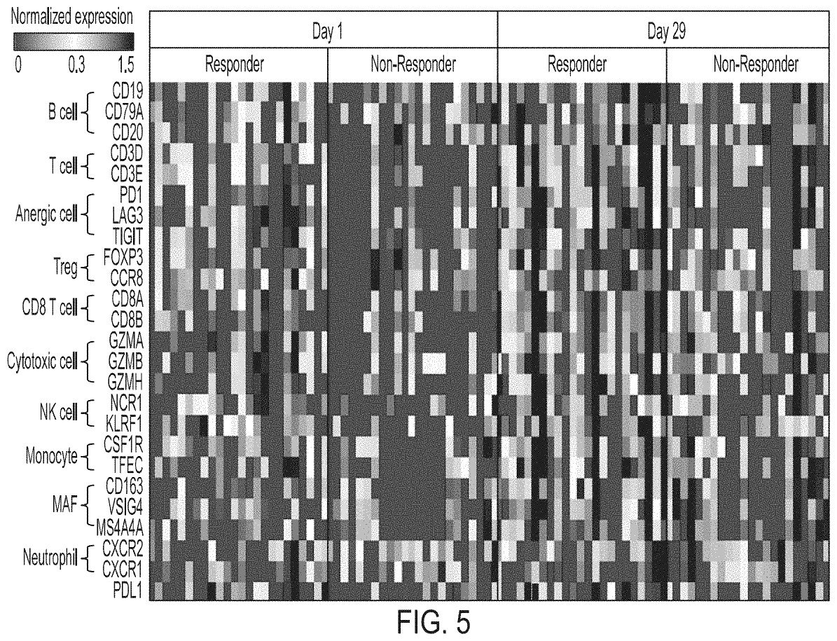

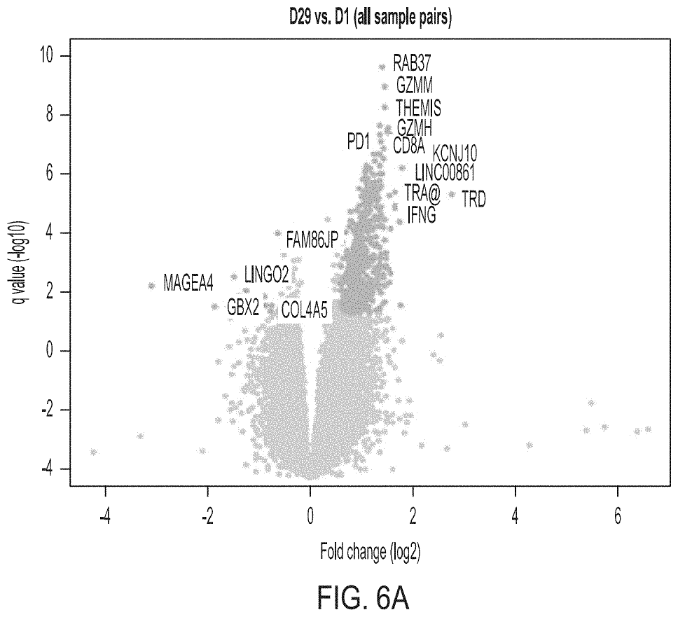

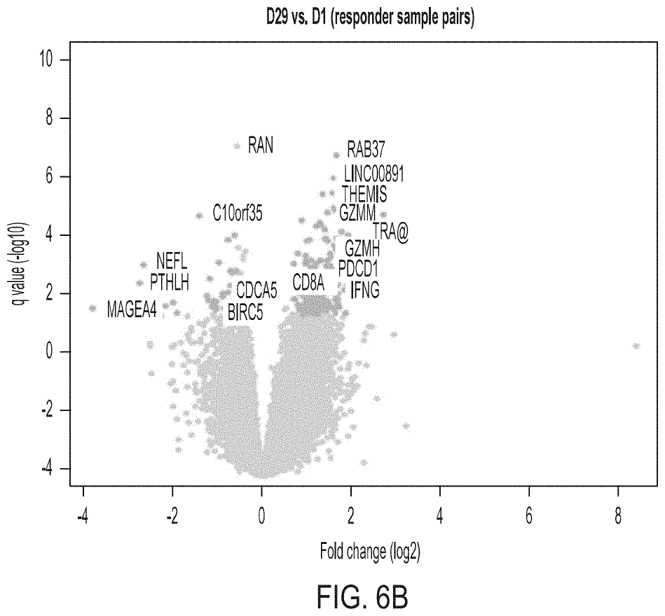

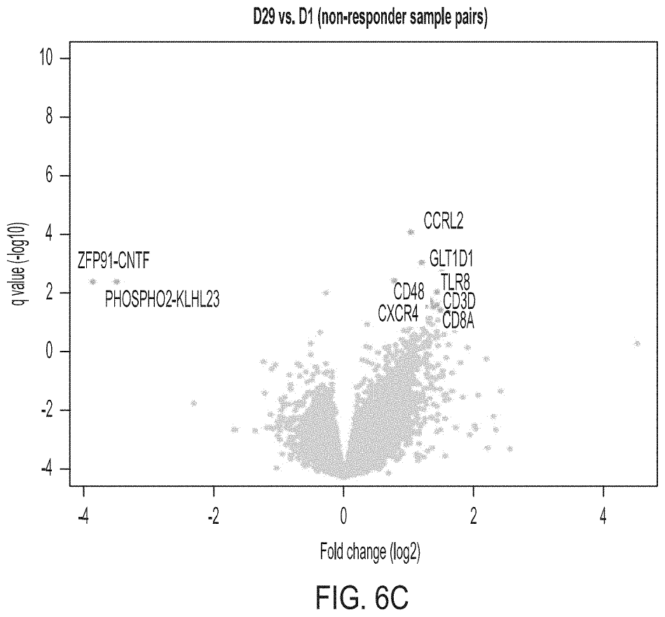

A treatment effect of Cemiplimab is an increase in expression of certain immune cell marker genes. As shown in FIG. 5, inferred immune marker gene expression in CSCC suggests Cemiplimab tends to increase the infiltration of immune cell subsets and this is more pronounced in responders. FIG. 6A shows differentially expressed genes determined by comparing the baseline gene expression data and the in-treatment gene expression for all patients (responder and non-responder). FIG. 6B shows differentially expressed genes determined by comparing the baseline gene expression data and the in-treatment gene expression for responders only. FIG. 6C shows differentially expressed genes determined by comparing the baseline gene expression data and the in-treatment gene expression for non-responders only. FIG. 6B and FIG. 6C indicate that responders have greater gene expression changes that non-responders.

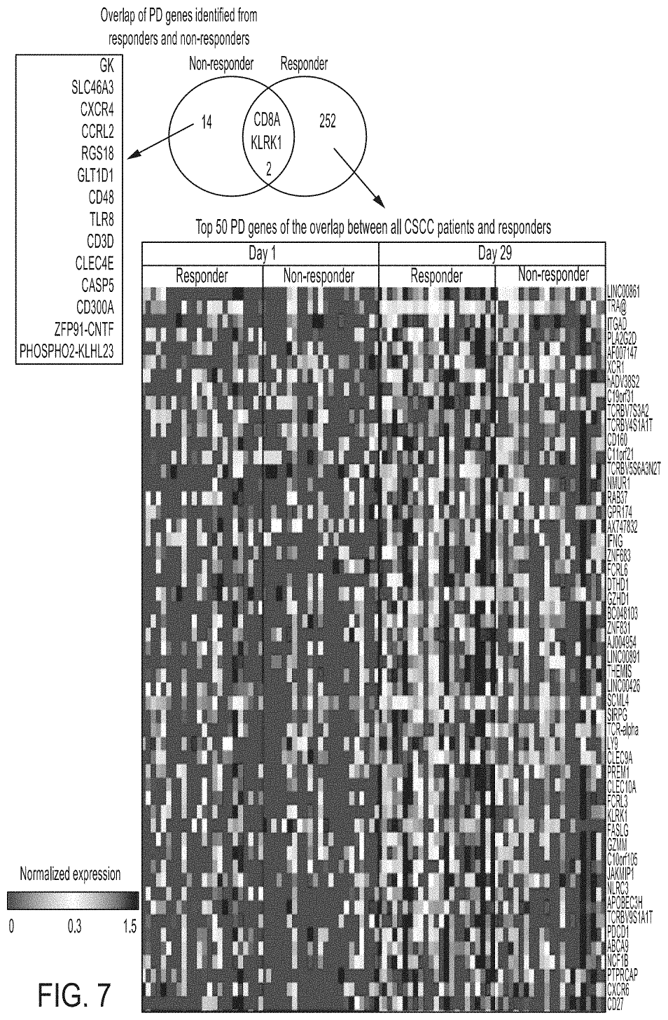

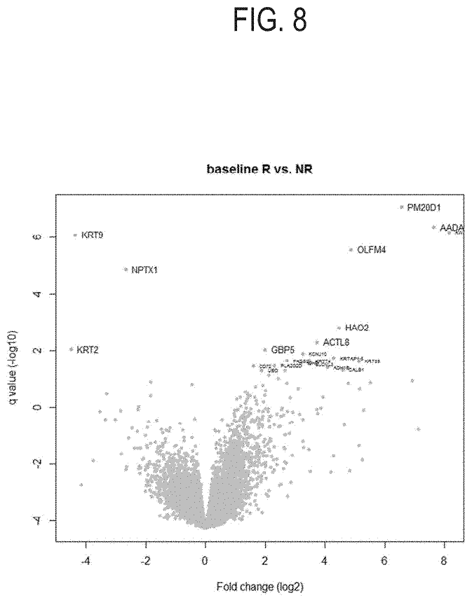

A comparison and analysis of the labeled baseline gene expression data and/or the labeled in-treatment gene expression data may determine one or more predictive genes. FIG. 7 shows the top 50 pharmcodynamic genes of the overlap between responders and non-responders. Of the 252 identified predictive genes for the responders and the 14 identified predictive genes for the non-responders, only 2 predictive genes were in common between responders and non-responders. As shown in FIG. 8, attempts to identify differentially expressed genes between baseline responders and baseline non-responders reveals very few statistically significant genes. The inability to determine sufficient predictive genes using the baseline gene expression data results from heterogeneous baseline samples, for example, tumor purity is often not quantified, and biopsy sites are often inconsistent between patients (e.g., skin, lung, head, neck, etc.). The result is identification of differentially expressed genes that are tissue specific. As a result, it is challenging to generate a baseline machine learning classifier according to the baseline gene expression data from this single study.

In an embodiment, curated disease agnostic gene set data from other studies involving the same drug/treatment may be analyzed to improve identification of predictive genes. The curated disease agnostic gene set data may be obtained from various sources, including recent publications. The curated disease agnostic gene set data may comprise multiple gene sets data, associated with different conditions (e.g., melanoma, breast cancer, lung cancer, ovarian cancer, etc.) and may be generated from various data types and/or platforms (e.g., bulk RNA-seq, single cell RNA-seq, NanoString, etc.). The curated disease agnostic gene set includes at least one gene in common with the CSCC data.

In the present example, the curated disease agnostic gene set was determined from one or more of the following publications:

[2018][Journal of ImmnoTherapy of Cancer][Turan T. et al][Immune oncology immune responsiveness and the theory of everything]

[2005][Richard D. Wood et al.][Human DNA repair genes, 2005]

[2017][Cell Reports][Charoentong P. et al.][Pan-cancer Immunogenomic Analyses Reveal Genotype-Immunophenotype Relationships and Predictors of Response to Checkpoint Blockade]

[2017][Wouter Hendrickx et al.][Identification of genetic determinants of breast cancer immune phenotypes by integrative genome scale analysis]

[2012][CancerImmImmunotherapy][Ji R. et al][An immune-active tumor microenvironment favors clinical response to ipilimumab]

[2013][JCO][Ulloba-Montoya F. et al][Predictive Gene Signature in MAGE-A3 Antigen-Specific Cancer Immunotherapy]

[2018][Nature Medicine][Peng Jiang][Signatures of T cell dysfunction and exclusion predict cancer immunotherapy response Nat Medicine Aug 2018]

[2018][Nature Medicine][Noam Auslander][Robust prediction of response to immune checkpoint blockade therapy in metastatic melanoma Nat Medicine Aug 2018]

[2018][NatMedicine][Savas.P. et al][Single-cell profiling of breast cancer T cells reveals a tissue-resident memory subset associated with improved prognosis]

[2018][Cell][Jerby-Amon L. et al.][Signature of T cell exclusion and ICI resistance Cancer Cell 2018]

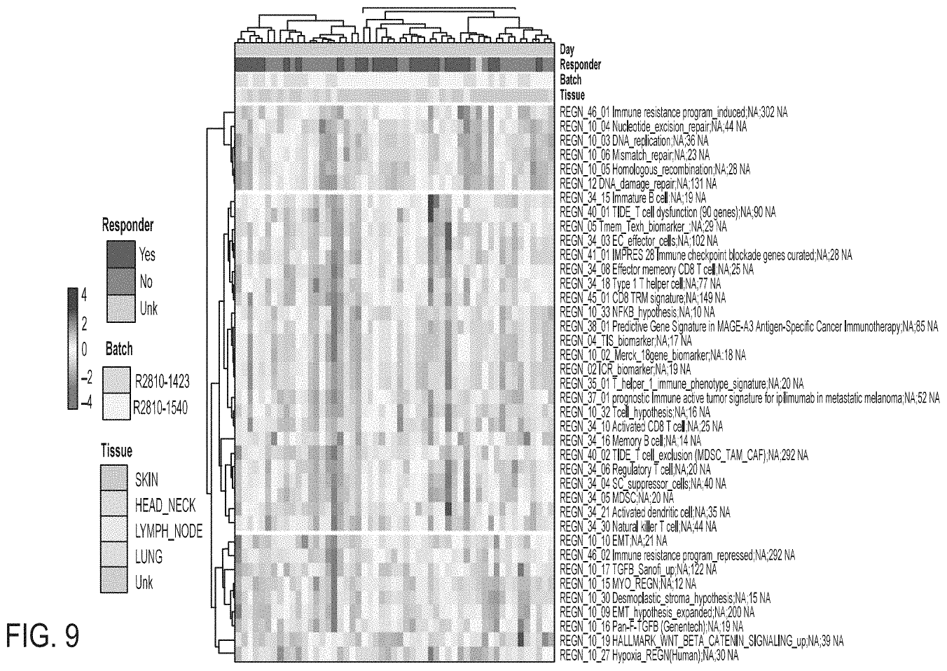

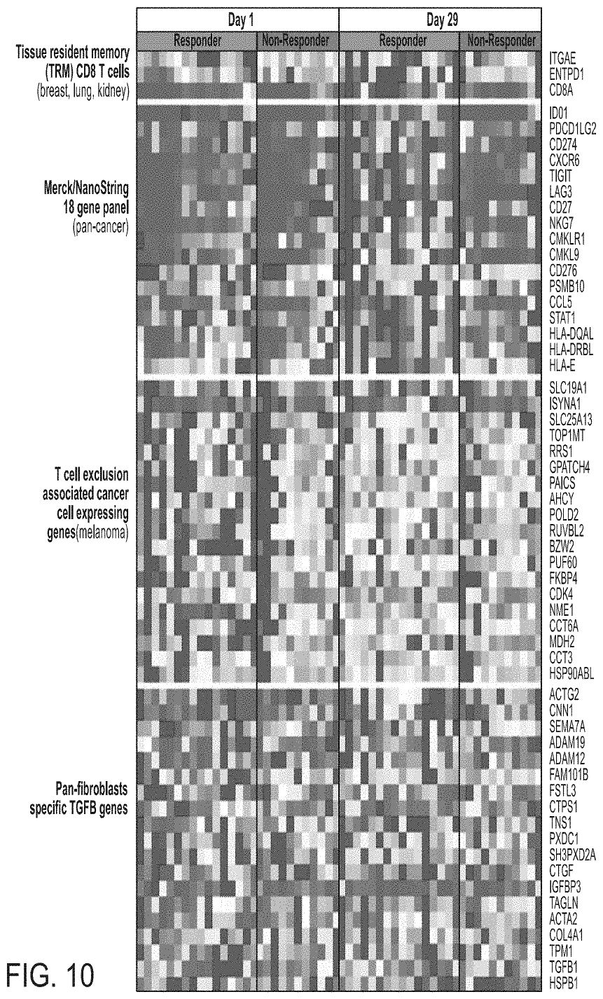

The curated disease agnostic gene set data are shown in FIG. 9. The curated disease agnostic gene set data may be categorized. For example, the disease agnostic gene set data may be categorized as immune cell type/function gene sets, tumor microenvironment component and signaling gene sets, and cancer cell proliferation and DNA repair gene sets. As with the originally obtained baseline gene expression data and in-treatment gene expression data, attempts to identify differentially expressed genes between baseline responders and baseline non-responders using the curated disease agnostic gene set data alone reveals very few statistically significant genes. FIG. 10 shows that predictive genes identified using the d curated disease agnostic gene set data alone only partially explains the clinical outcomes of CSCC cohorts.

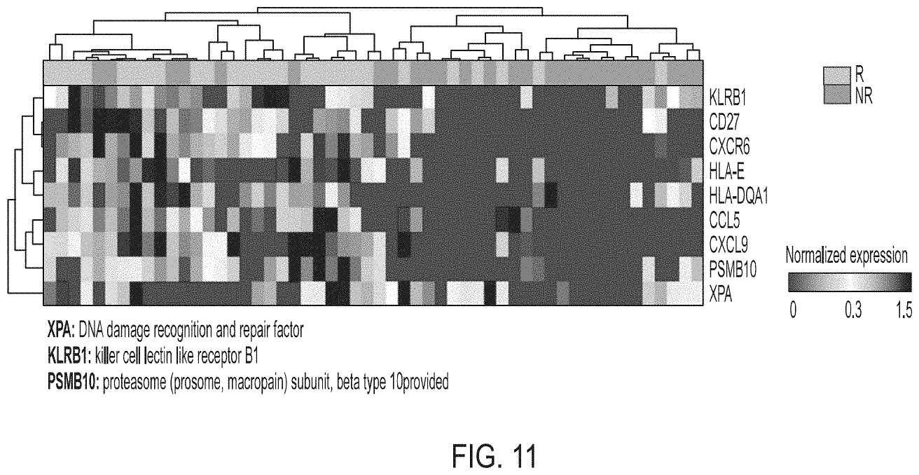

The machine learning techniques described herein were applied to the baseline gene expression data and the curated disease agnostic gene set data. FIG. 11 shows a top performing gene signature generated using the machine learning techniques described above. FIG. 11 shows the normalized gene expression of the top performing predictive gene signature. The patient samples are identified at the top of FIG. 11 as R (Responder) or NR (Non-Responder). FIG. 11 shows that the patients having higher expression (dark red in FIG. 11) of the top performing predictive gene signature have a higher probability of being a Responder (Orange), while the patients having lower expression (dark blue in FIG. 11) of the top performing predictive gene signature have a higher probability of being a Non-Responder (skyblue).

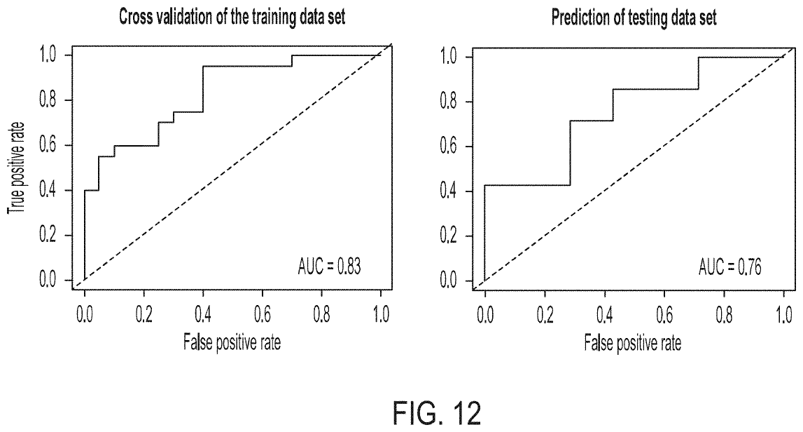

FIG. 12 shows the performance of the gene signature in classifying the patients during the machine learning model training based on cross validation and as applied to the testing data set. The Area Under Curve (AUC) of Receiver Operation Curve (ROC) represents the performance of a classification method.

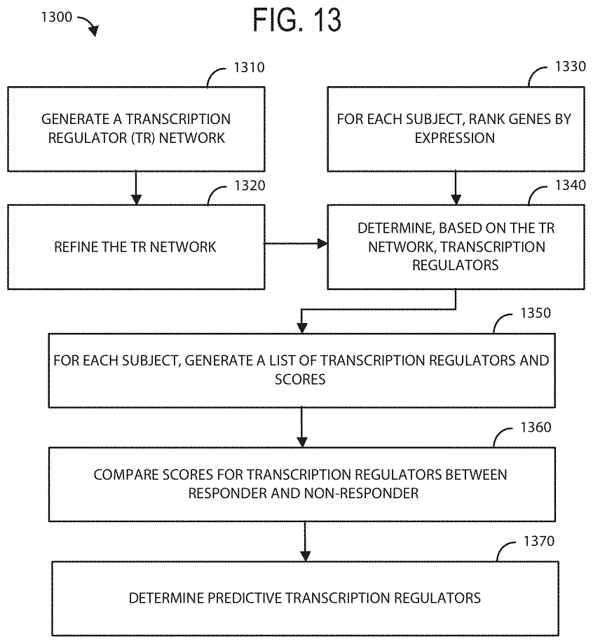

In an embodiment, FIG. 13 shows a method 1300 for a systems biology approach to identify predictive transcription regulator genes.

A transcription regulator network may be generated at 1310. Transcription regulator data may be obtained from the Gene Ontology (GO) resource. The transcription regulator data may comprise a list of genes identified as transcription regulator genes and any genes that could affect the transcription of other genes as annotated in GO. The transcription regulator network may be generated, for example, by ARACNE (Algorithm for the Reconstruction of Gene Regulatory Networks in a Mammalian Cellular Context). The transcription regulator network may comprise a plurality of nodes, wherein each node is a gene (transcription regulator gene or target gene), and a plurality of edges, wherein an edge between two nodes may indicate a relationship. The relationship may indicate transcription regulator genes associated with one or more target genes. The relationship may comprise, for example, “is a transcription regulator of” or “transcription is regulated by.” In an embodiment, the baseline gene expression data may be used to filter the transcription regulator data. Genes present in both the gene expression data and in the target genes of the transcription regulator data may be identified. The identified genes and the associated transcription regulator genes may be used to generate the transcription network. In an embodiment, a mutual information-based method to determine a relationship between a transcription regulator gene and any other gene in the gene expression data so that the transcription network connecting the transcription regulator gene and their target genes is constructed.

The transcription regulator network may be refined at 1320. Refining the transcription regulator network may comprise removing one or more edges that likely occurred by chance. Refining may be performed based on number of samples in the gene expression data that used to construct the network and computation of a probability of each network connection being discovered reliably given the sample number. For example, the network connections may be randomly permuted and a probability of that network connection being observed may be determined. Any network connections with a probability that is not statistically significant (e.g., higher than a p-value) may be removed.

Sequentially, or in parallel, at 1330, the genes for each subject in the baseline gene expression data may be ranked by expression as derived from the baseline gene expression data. At 1340, transcription regulator genes that target the ranked genes may be determined based on the transcription regulator network and the ranked list of genes. The transcription network may be traversed to identify a node associated with a set of target genes that are also found in the ranked list of genes. An edge may be determined that flows from that node to identify a transcription regulator gene associated with the set of target genes.

At 1350, for each subject in the baseline gene expression data, an enrichment score for each transcription regulator gene associated with that subject may be determined. The enrichment score for the transcription regulator gene may be based on the rank of the gene expression of its transcription target genes identified in the transcription network.

The enrichment scores for each of the of the transcription regulator genes may be compared at 1360. For example, a ratio of enrichment scores for a transcription regulator gene between baseline responder non-responder may be determined.

One or more predictive transcription regulator genes may be determined at 1370. The one or more predictive transcription regulator genes may be determined by assessing the statistical significance of the ratio of enrichment scores for a given transcription regulator gene. Transcription regulator genes having a ratio of enrichment scores that is statistically significant may be identified as a predictive transcription regulator gene.

The one or more predictive transcription regulator genes may be used to identify a candidate for a therapeutic treatment. Baseline gene expression data may be obtained from a new subject. The baseline gene expression data may be ranked, and target genes of the predictive transcription regulator genes based on the network was collected and then an enrichment score was computed to identify the activity of the predictive transcription regulator genes. If the subject possess high enrichment score of the one or more predictive transcription regulator genes, then the subject is a candidate for the therapeutic treatment.

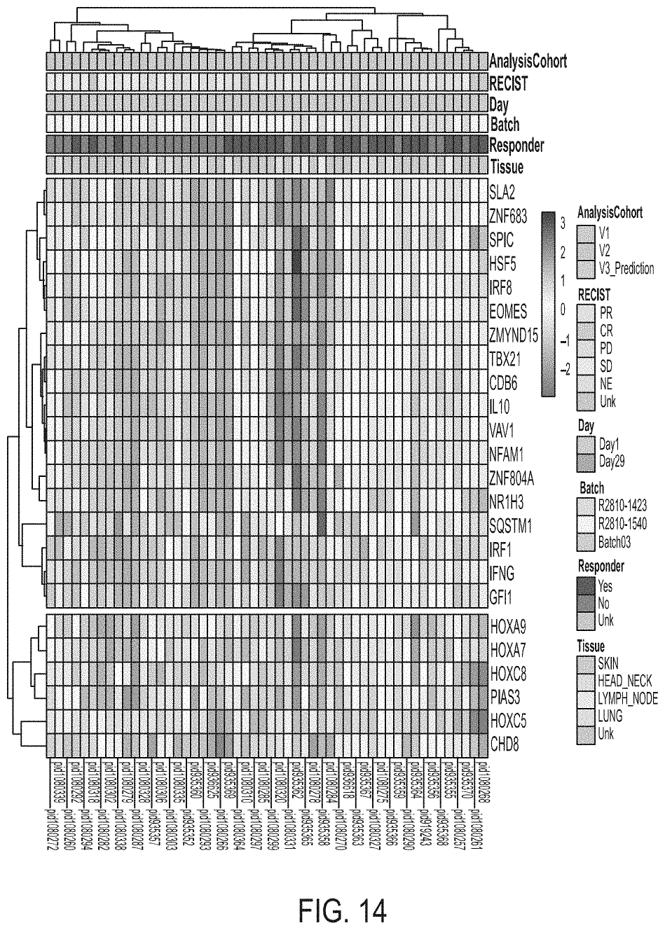

FIG. 14 shows example top predictive transcription regulator genes and their enrichment score as determined using the CSCC cohort baseline samples described previously.

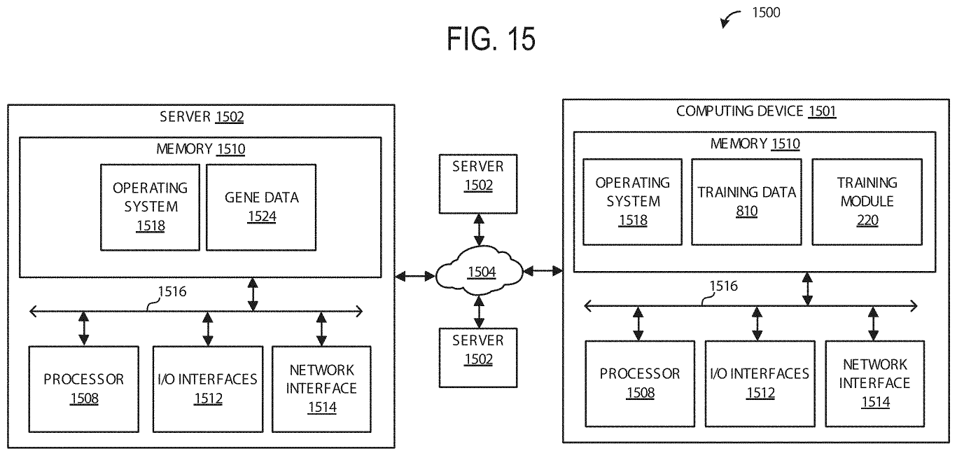

FIG. 15 is a block diagram depicting an environment 1500 comprising non-limiting examples of a computing device 1501 and a server 1502 connected through a network 1504. In an aspect, some or all steps of any described method may be performed on a computing device as described herein. The computing device 1501 can comprise one or multiple computers configured to store one or more of the training module 220, training data 210 (e.g., labeled baseline gene expression data, labeled in-treatment gene expression data, and/or curated disease agnostic gene set data), and the like. The server 1402 can comprise one or multiple computers configured to store gene data 1524 (e.g., curated disease agnostic gene set data). Multiple servers 1502 can communicate with the computing device 1501 via the through the network 1504.

The computing device 1501 and the server 1502 can be a digital computer that, in terms of hardware architecture, generally includes a processor 1508, memory system 1510, input/output (I/O) interfaces 1512, and network interfaces 1514. These components (1508, 1510, 1512, and 1514) are communicatively coupled via a local interface 1516. The local interface 1516 can be, for example, but not limited to, one or more buses or other wired or wireless connections, as is known in the art. The local interface 1516 can have additional elements, which are omitted for simplicity, such as controllers, buffers (caches), drivers, repeaters, and receivers, to enable communications. Further, the local interface may include address, control, and/or data connections to enable appropriate communications among the aforementioned components.

The processor 1508 can be a hardware device for executing software, particularly that stored in memory system 1510. The processor 1508 can be any custom made or commercially available processor, a central processing unit (CPU), an auxiliary processor among several processors associated with the computing device 1501 and the server 1502, a semiconductor-based microprocessor (in the form of a microchip or chip set), or generally any device for executing software instructions. When the computing device 1501 and/or the server 1502 is in operation, the processor 1508 can be configured to execute software stored within the memory system 1510, to communicate data to and from the memory system 1510, and to generally control operations of the computing device 1501 and the server 1502 pursuant to the software.

The I/O interfaces 1512 can be used to receive user input from, and/or for providing system output to, one or more devices or components. User input can be provided via, for example, a keyboard and/or a mouse. System output can be provided via a display device and a printer (not shown). I/O interfaces 1512 can include, for example, a serial port, a parallel port, a Small Computer System Interface (SCSI), an infrared (IR) interface, a radio frequency (RF) interface, and/or a universal serial bus (USB) interface.

The network interface 1514 can be used to transmit and receive from the computing device 1501 and/or the server 1502 on the network 1504. The network interface 1514 may include, for example, a 10BaseT Ethernet Adaptor, a 100BaseT Ethernet Adaptor, a LAN PHY Ethernet Adaptor, a Token Ring Adaptor, a wireless network adapter (e.g., WiFi, cellular, satellite), or any other suitable network interface device. The network interface 1514 may include address, control, and/or data connections to enable appropriate communications on the network 1504.

The memory system 1510 can include any one or combination of volatile memory elements (e.g., random access memory (RAM, such as DRAM, SRAM, SDRAM, etc.)) and nonvolatile memory elements (e.g., ROM, hard drive, tape, CDROM, DVDROM, etc.). Moreover, the memory system 1510 may incorporate electronic, magnetic, optical, and/or other types of storage media. Note that the memory system 1510 can have a distributed architecture, where various components are situated remote from one another, but can be accessed by the processor 1508.

The software in memory system 1510 may include one or more software programs, each of which comprises an ordered listing of executable instructions for implementing logical functions. In the example of FIG. 15, the software in the memory system 1510 of the computing device 1501 can comprise the training module 220 (or subcomponents thereof), the training data 220, and a suitable operating system (O/S) 1518. In the example of FIG. 15, the software in the memory system 1510 of the server 1502 can comprise, the gene data 1524, and a suitable operating system (O/S) 1518. The operating system 1518 essentially controls the execution of other computer programs and provides scheduling, input-output control, file and data management, memory management, and communication control and related services.

For purposes of illustration, application programs and other executable program components such as the operating system 1518 are illustrated herein as discrete blocks, although it is recognized that such programs and components can reside at various times in different storage components of the computing device 1501 and/or the server 1502. An implementation of the training module 220 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

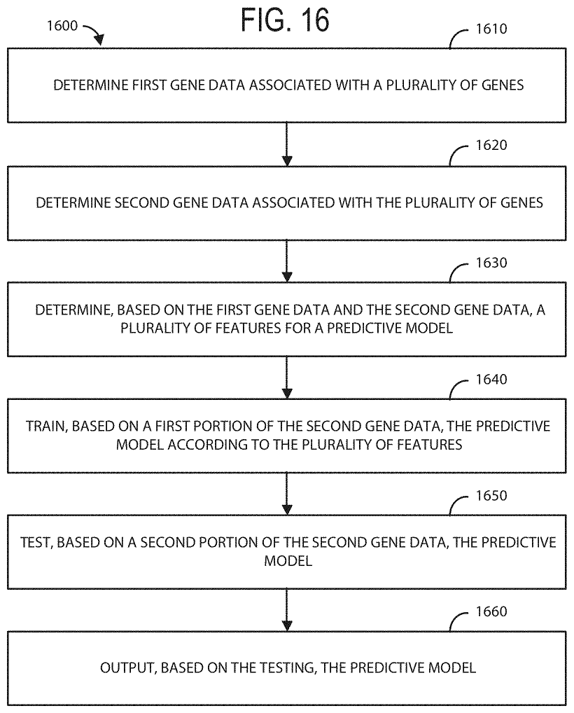

In an embodiment, the training module 220 may be configured to perform a method 1600, shown in FIG. 16. The method 1600 may be performed in whole or in part by a single computing device, a plurality of electronic devices, and the like. The method 1600 may comprise determining first gene data associated with a plurality of genes at 1610. The first gene data may be comprised of gene data from a plurality of different data sets. The first gene data may be retrieved from a public data source and the plurality of genes comprise one or more of an immune cell type/function gene set, a tumor microenvironment component and signaling gene set, or a cancer cell proliferation and DNA repair gene set.

The method 1600 may comprise determining second gene data associated with the plurality of genes at 1620. The plurality of genes may be sequenced from a plurality of tumor samples. Each tumor sample of the plurality of tumor samples may be labeled as a responder or a non-responder. Determining the second gene data associated with the plurality of genes may comprise determining baseline gene expression levels for each tumor associated with the plurality of tumor samples, treating each tumor associated with the plurality of tumor samples with a therapeutic, determining, post-treatment, which tumors associated with the plurality of tumor samples are responders or non-responders to the therapeutic, labeling the baseline gene expression levels for each tumor associated with the plurality of tumor samples, as responder or non-responder, and generating, based on the labeled baseline gene expression levels, the second gene data.

The method 1600 may comprise determining, based on the first gene data and the second gene data, a plurality of features for a predictive model at 1630. Determining, based on the first gene data and the second gene data, the plurality of features for the predictive model may comprise determining, from the first gene data, genes present in two or more of the plurality of different data sets as a first set of candidate genes, determining, from the second gene data, genes of the first set of candidate genes expressed at greater than or equal to 2 Transcripts Per Million (TPM) in at least half of the plurality of tumor samples as a second set of candidate genes, and determining, from the second gene data, genes of the second set of candidate genes with a statistically significant increase in expression level between responders and non-responders as a third set of candidate genes, wherein the plurality of features comprises the third set of candidate genes. Determining, based on the first gene data and the second gene data, the plurality of features for the predictive model may comprise determining, for the third set of candidate genes, a tumor mutational burden (TMB) value for each of the plurality of tumors associated with the third set of candidate genes and determining, based on the TMB values, a fourth set of candidate genes, wherein the plurality of features comprises the fourth set of candidate genes.

The method 1600 may comprise training, based on a first portion of the second gene data, the predictive model according to the plurality of features at 1640. Training, based on the first portion of the second gene data, the predictive model according to the plurality of features results in determining a gene signature indicative of a responder.

The method 1600 may comprise testing, based on a second portion of the second gene data, the predictive model at 1650. The method 1600 may comprise outputting, based on the testing, the predictive model at 1660.

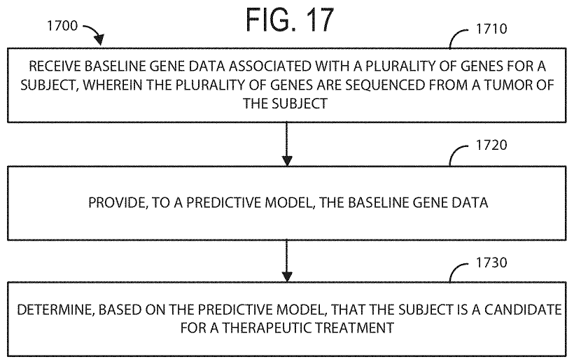

In an embodiment, the training module 220 may be configured to perform a method 1700, shown in FIG. 17. The method 1700 may be performed in whole or in part by a single computing device, a plurality of electronic devices, and the like. The method 1700 may comprise receiving baseline gene data associated with a plurality of genes for a subject at 1710. The plurality of genes may be sequenced from a tumor of the subject. The method 1700 may comprise providing, to a predictive model, the baseline gene data at 1720. The method 1700 may comprise determining, based on the predictive model, that the subject is a candidate for a therapeutic treatment at 1730. The method 1700 may further comprise treating the subject with the therapeutic treatment.

The method 1700 may further comprise training the predictive model.

Training the predictive model may comprise determining first gene data associated with the plurality of genes, determining second gene data associated with the plurality of genes, wherein the plurality of genes are sequenced from a plurality of tumor samples, wherein each tumor sample of the plurality of tumor samples is labeled as a responder or a non-responder, determining, based on the first gene data and the second gene data, a plurality of features for the predictive model, training, based on a first portion of the second gene data, the predictive model according to the plurality of features, testing, based on a second portion of the second gene data, the predictive model, and outputting, based on the testing, the predictive model.

The first gene data may be retrieved from a public data source and the plurality of genes may comprise one or more of an immune cell type/function gene set, a tumor microenvironment component and signaling gene set, or a cancer cell proliferation and DNA repair gene set. The first gene data may be comprised of gene data from a plurality of different data sets.

Determining, based on the first gene data and the second gene data, the plurality of features for the predictive model may comprise determining, from the first gene data, genes present in two or more of the plurality of different data sets as a first set of candidate genes, determining, from the second gene data, genes of the first set of candidate genes expressed at greater than or equal to 2 Transcripts Per Million (TPM) in at least half of the plurality of tumor samples as a second set of candidate genes, and determining, from the second gene data, genes of the second set of candidate genes with a statistically significant increase in expression level between responders and non-responders as a third set of candidate genes, wherein the plurality of features comprises the third set of candidate genes.

Determining, based on the first gene data and the second gene data, the plurality of features for the predictive model may comprise determining, for the third set of candidate genes, a tumor mutational burden (TMB) value for each of the plurality of tumors associated with the third set of candidate genes and determining, based on the TMB values, a fourth set of candidate genes, wherein the plurality of features comprises the fourth set of candidate genes.

Determining the second gene data associated with the plurality of genes may comprise determining baseline gene expression levels for each tumor associated with the plurality of tumor samples, treating each tumor associated with the plurality of tumor samples with a therapeutic, determining, post-treatment, which tumors associated with the plurality of tumor samples are responders or non-responders to the therapeutic, labeling the baseline gene expression levels for each tumor associated with the plurality of tumor samples, as responder or non-responder, and generating, based on the labeled baseline gene expression levels, the second gene data.

Training, based on the first portion of the second gene data, the predictive model according to the plurality of features results in determining a gene signature indicative of a responder.

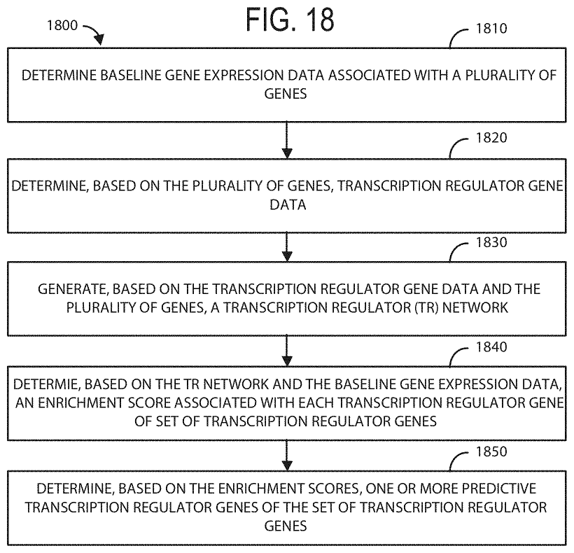

In an embodiment, the training module 220 may be configured to perform a method 1800, shown in FIG. 18. The method 1800 may be performed in whole or in part by a single computing device, a plurality of electronic devices, and the like. The method 1800 may comprise determining baseline gene expression data associated with a plurality of genes at 1810. The plurality of genes may be associated with a plurality of tumor samples and each tumor sample of the plurality of tumor samples may be labeled as a responder or a non-responder to a therapeutic/treatment. Determining baseline gene expression data may comprise determining baseline gene expression levels for each tumor associated with the plurality of tumor samples, treating each tumor associated with the plurality of tumor samples with a therapeutic, determining, post-treatment, which tumors associated with the plurality of tumor samples are responders or non-responders to the therapeutic, labeling the baseline gene expression levels for each tumor associated with the plurality of tumor samples, as responder or non-responder, and generating, based on the labeled baseline gene expression levels, the baseline gene expression data.

The method 1800 may comprise determining, based on the plurality of genes, transcription regulator gene data at 1820. Determining, based on the plurality of genes, the transcription regulator gene data may comprise querying a gene ontology database for any gene having a transcription function, determining, based on the query, one or more transcription regulation genes and associated target genes, and generating, based on the one or more transcription regulation genes and the associated target genes, the transcription regulator gene data.

The method 1800 may comprise generating, based on the transcription regulator gene data and the plurality of genes, a transcription regulator (TR) network at 1830. Generating, based on the transcription regulator gene data and the plurality of genes, the TR network may comprise generating a plurality of nodes, wherein each node of the plurality of nodes represents either a transcription regulator gene or a target gene, connecting two or more of the plurality of nodes with one or more edges, wherein each edge represents a relationship between a transcription regulator gene and a target gene, and storing the plurality of nodes and the one or more edges as the TR network. The relationship may indicate that the transcription regulator gene regulates transcription of the target gene. The method 1800 may further comprise refining the TR network. Refining the TR network may comprise deleting one or more edges that likely occurred by chance.

The method 1800 may comprise determining, based on the TR network and the baseline gene expression data, an enrichment score associated with each transcription regulator gene of set of transcription regulator genes at 1840. The enrichment score associated with each transcription regulator gene of set of transcription regulator genes may be based on one or more enrichment scores associated with one or more genes in the baseline gene expression data associated with the transcription regulator gene.

The method 1800 may comprise determining, based on the enrichment scores, one or more predictive transcription regulator genes of the set of transcription regulator genes at 1850. Determining, based on the enrichment scores, the one or more predictive transcription regulator genes of the set of transcription regulator genes may comprise determining an enrichment score ratio of responder to non-responder for each transcriptional regulator gene of the set of transcription regulator genes and determining transcriptional regulator genes of the set of transcription regulator genes with a statistically significant association with responders as the one or more predictive transcription regulator genes.

The method 1800 may further comprise determining additional baseline gene expression data for a subject, determining a presence of the one or more predictive transcription regulator genes in the additional baseline gene expression data, and determining, based on the presence of the one or more predictive transcription regulator genes in the additional baseline gene expression data, that the subject is a candidate for a therapeutic treatment.

Embodiment 1: A method comprising: determining first gene data associated with a plurality of genes, determining second gene data associated with the plurality of genes, wherein the plurality of genes are sequenced from a plurality of tumor samples, wherein each tumor sample of the plurality of tumor samples is labeled as a responder or a non-responder, determining, based on the first gene data and the second gene data, a plurality of features for a predictive model, training, based on a first portion of the second gene data, the predictive model according to the plurality of features, testing, based on a second portion of the second gene data, the predictive model, and outputting, based on the testing, the predictive model.

Embodiment 2: The embodiment as in any one of the preceding embodiments wherein determining the first gene data associated with a plurality of genes comprises retrieving the first gene data from a public data source.

Embodiment 3: The embodiment as in any one of the preceding embodiments, wherein the plurality of genes comprise one or more of an immune cell type/function gene set, a tumor microenvironment component and signaling gene set, or a cancer cell proliferation and DNA repair gene set.

Embodiment 4: The embodiment as in any one of the preceding embodiments, wherein determining the first gene data associated with the plurality of genes comprises: determining, based on the second gene data, the plurality of genes, determining, based on the plurality of genes, one or more gene data sets that comprise at least one gene of the plurality of genes, and generating, based on the one or more gene data sets, the first gene data.

Embodiment 5: The embodiment as in any one of the preceding embodiments wherein the first gene data is comprised of gene data from a plurality of different gene data sets.

Embodiment 6: The embodiment as in any one of the preceding embodiments wherein determining the second gene data associated with the plurality of genes comprises: determining baseline gene expression levels for each tumor associated with the plurality of tumor samples, treating each tumor associated with the plurality of tumor samples with a therapeutic, determining, post-treatment, which tumors associated with the plurality of tumor samples are responders or non-responders to the therapeutic, labeling the baseline gene expression levels for each tumor associated with the plurality of tumor samples, as responder or non-responder, and generating, based on the labeled baseline gene expression levels, the second gene data.

Embodiment 7: The embodiment as in any one of the embodiments 5-6 wherein determining, based on the first gene data and the second gene data, the plurality of features for the predictive model comprises: determining, from the first gene data, genes present in two or more of the plurality of different gene data sets as a first set of candidate genes, determining, from the second gene data, genes of the first set of candidate genes expressed at greater than or equal to 2 Transcripts Per Million (TPM) in at least half of the plurality of tumor samples as a second set of candidate genes, and determining, from the second gene data, genes of the second set of candidate genes with a statistically significant increase in expression level between responders and non-responders as a third set of candidate genes, wherein the plurality of features comprises the third set of candidate genes.

Embodiment 8: The embodiment as in any one of the embodiments 5-7 wherein determining, based on the first gene data and the second gene data, the plurality of features for the predictive model comprises: determining, for the third set of candidate genes, a tumor mutational burden (TMB) value for each of the plurality of tumors associated with the third set of candidate genes, and determining, based on the TMB values, a fourth set of candidate genes, wherein the plurality of features comprises the fourth set of candidate genes.

Embodiment 9: The embodiment as in any one of the preceding embodiments wherein training, based on the first portion of the second gene data, the predictive model according to the plurality of features results in determining a gene signature indicative of a responder.

Embodiment 10: A method comprising: receiving baseline gene data associated with a plurality of genes for a subject, wherein the plurality of genes are sequenced from a tumor of the subject, providing, to a predictive model, the baseline gene data, and determining, based on the predictive model, that the subject is a candidate for a therapeutic treatment.

Embodiment 11: The embodiment as in the embodiment 10 further comprising training the predictive model.

Embodiment 12: The embodiment as in any one of the embodiments 10-11 further comprising training the predictive model.

Embodiment 13: The embodiment as in any one of the embodiments 10-12, wherein training the predictive model comprises: determining first gene data associated with the plurality of genes, determining second gene data associated with the plurality of genes, wherein the plurality of genes are sequenced from a plurality of tumor samples, wherein each tumor sample of the plurality of tumor samples is labeled as a responder or a non-responder, determining, based on the first gene data and the second gene data, a plurality of features for the predictive model, training, based on a first portion of the second gene data, the predictive model according to the plurality of features, testing, based on a second portion of the second gene data, the predictive model, and outputting, based on the testing, the predictive model.

Embodiment 14: The embodiment as in the embodiment 13 wherein determining the first gene data associated with the plurality of genes comprises: determining, based on the second gene data, the plurality of genes, determining, based on the plurality of genes, one or more gene data sets that comprise at least one gene of the plurality of genes, and generating, based on the one or more gene data sets, the first gene data.

Embodiment 15: The embodiment as in the embodiments 13-14 wherein the first gene data is comprised of gene data from a plurality of different gene data sets.

Embodiment 16: The embodiment as in the embodiments 13-15 wherein determining the second gene data associated with the plurality of genes comprises: determining baseline gene expression levels for each tumor associated with the plurality of tumor samples, treating each tumor associated with the plurality of tumor samples with a therapeutic, determining, post-treatment, which tumors associated with the plurality of tumor samples are responders or non-responders to the therapeutic, labeling the baseline gene expression levels for each tumor associated with the plurality of tumor samples, as responder or non-responder, and generating, based on the labeled baseline gene expression levels, the second gene data.

Embodiment 17: The embodiment as in the embodiments 14-16 wherein determining, based on the first gene data and the second gene data, the plurality of features for the predictive model comprises: determining, from the first gene data, genes present in two or more of the plurality of different gene data sets as a first set of candidate genes, determining, from the second gene data, genes of the first set of candidate genes expressed at greater than or equal to 2 Transcripts Per Million (TPM) in at least half of the plurality of tumor samples as a second set of candidate genes, and determining, from the second gene data, genes of the second set of candidate genes with a statistically significant increase in expression level between responders and non-responders as a third set of candidate genes, wherein the plurality of features comprises the third set of candidate genes.

Embodiment 18: The embodiment as in the embodiments 14-17 wherein determining, based on the first gene data and the second gene data, the plurality of features for the predictive model comprises: determining, for the third set of candidate genes, a tumor mutational burden (TMB) value for each of the plurality of tumors associated with the third set of candidate genes, and determining, based on the TMB values, a fourth set of candidate genes, wherein the plurality of features comprises the fourth set of candidate genes.

Embodiment 19: The embodiment as in the embodiments 10-18 wherein training, based on the first portion of the second gene data, the predictive model according to the plurality of features results in determining a gene signature indicative of a responder.

Embodiment 20: A method comprising: determining baseline gene expression data associated with a plurality of genes, wherein the plurality of genes are associated with a plurality of tumor samples, wherein each tumor sample of the plurality of tumor samples is labeled as a responder or a non-responder, determining, based on the plurality of genes, transcription regulator gene data, generating, based on the transcription regulator gene data and the plurality of genes, a transcription regulator (TR) network, determining, based on the TR network and the baseline gene expression data, an enrichment score associated with each transcription regulator gene of set of transcription regulator genes, and determining, based on the enrichment scores, one or more predictive transcription regulator genes of the set of transcription regulator genes.

Embodiment 21: The embodiment as in the embodiment 20 wherein determining baseline gene expression data comprises: determining baseline gene expression levels for each tumor associated with the plurality of tumor samples, treating each tumor associated with the plurality of tumor samples with a therapeutic, determining, post-treatment, which tumors associated with the plurality of tumor samples are responders or non-responders to the therapeutic, labeling the baseline gene expression levels for each tumor associated with the plurality of tumor samples, as responder or non-responder, and generating, based on the labeled baseline gene expression levels, the baseline gene expression data.

Embodiment 22: The embodiment as in any one of the embodiments 20-21 wherein determining, based on the plurality of genes, the transcription regulator gene data comprises: querying a gene ontology database for any gene having a transcription function, determining, based on the query, one or more transcription regulation genes and associated target genes, and generating, based on the one or more transcription regulation genes and the associated target genes, the transcription regulator gene data.

Embodiment 23: The embodiment as in any one of the embodiments 20-22 wherein generating, based on the transcription regulator gene data and the plurality of genes, the TR network comprises: generating a plurality of nodes, wherein each node of the plurality of nodes represents either a transcription regulator gene or a target gene, connecting two or more of the plurality of nodes with one or more edges, wherein each edge represents a relationship between a transcription regulator gene and a target gene, and storing the plurality of nodes and the one or more edges as the TR network.

Embodiment 24: The embodiment as in any one of the embodiments 20-23 wherein the relationship indicates that the transcription regulator gene regulates transcription of the target gene.

Embodiment 25: The embodiment as in any one of the embodiments 20-24 further comprising refining the TR network.

Embodiment 26: The embodiment as in the embodiment 25 wherein refining the TR network comprises deleting one or more edges that likely occurred by chance.

Embodiment 27: The embodiment as in any one of the embodiments 20-26 wherein the enrichment score associated with each transcription regulator gene of set of transcription regulator genes is based on one or more enrichment scores associated with one or more genes in the baseline gene expression data associated with the transcription regulator gene.

Embodiment 28: The embodiment as in any one of the embodiments 20-27 wherein determining, based on the enrichment scores, the one or more predictive transcription regulator genes of the set of transcription regulator genes comprises: determining an enrichment score ratio of responder to non-responder for each transcriptional regulator gene of the set of transcription regulator genes, and determining transcriptional regulator genes of the set of transcription regulator genes with a statistically significant association with responders as the one or more predictive transcription regulator genes.

Embodiment 29: The embodiment as in any one of the embodiments 20-28 further comprising: determining additional baseline gene expression data for a subject, determining a presence of the one or more predictive transcription regulator genes in the additional baseline gene expression data, and determining, based on the presence of the one or more predictive transcription regulator genes in the additional baseline gene expression data, that the subject is a candidate for a therapeutic treatment.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A method comprising:
determining first gene data associated with a plurality of genes;
determining second gene data associated with the plurality of genes, wherein the plurality of genes are sequenced from a plurality of tumor samples, wherein each tumor sample of the plurality of tumor samples is labeled as a responder or a non-responder;
determining, based on the first gene data and the second gene data, a plurality of features for a predictive model;
training, based on a first portion of the second gene data, the predictive model according to the plurality of features;
testing, based on a second portion of the second gene data, the predictive model; and
outputting, based on the testing, the predictive model.

2. The method of claim 1, wherein determining the first gene data associated with a plurality of genes comprises retrieving the first gene data from a public data source.

3. The method of claim 1, wherein the plurality of genes comprise one or more of an immune cell type/function gene set, a tumor microenvironment component and signaling gene set, or a cancer cell proliferation and DNA repair gene set.

4. The method of claim 1, wherein determining the first gene data associated with the plurality of genes comprises:
determining, based on the second gene data, the plurality of genes;
determining, based on the plurality of genes, one or more gene data sets that comprise at least one gene of the plurality of genes; and
generating, based on the one or more gene data sets, the first gene data.

5. The method of claim 1, wherein the first gene data is comprised of gene data from a plurality of different gene data sets.

6. The method of claim 1, wherein determining the second gene data associated with the plurality of genes comprises:
determining baseline gene expression levels for each tumor associated with the plurality of tumor samples;
treating each tumor associated with the plurality of tumor samples with a therapeutic;
determining, post-treatment, which tumors associated with the plurality of tumor samples are responders or non-responders to the therapeutic;
labeling the baseline gene expression levels for each tumor associated with the plurality of tumor samples, as responder or non-responder; and
generating, based on the labeled baseline gene expression levels, the second gene data.

7. The method of claim 5, wherein determining, based on the first gene data and the second gene data, the plurality of features for the predictive model comprises:
determining, from the first gene data, genes present in two or more of the plurality of different gene data sets as a first set of candidate genes;
determining, from the second gene data, genes of the first set of candidate genes expressed at greater than or equal to 2 Transcripts Per Million (TPM) in at least half of the plurality of tumor samples as a second set of candidate genes; and
determining, from the second gene data, genes of the second set of candidate genes with a statistically significant increase in expression level between responders and non-responders as a third set of candidate genes,
wherein the plurality of features comprises the third set of candidate genes.

8. The method of claim 7, wherein determining, based on the first gene data and the second gene data, the plurality of features for the predictive model comprises:
determining, for the third set of candidate genes, a tumor mutational burden (TMB) value for each of the plurality of tumors associated with the third set of candidate genes; and
determining, based on the TMB values, a fourth set of candidate genes, wherein the plurality of features comprises the fourth set of candidate genes.

9. The method of claim 1, wherein training, based on the first portion of the second gene data, the predictive model according to the plurality of features results in determining a gene signature indicative of a responder.

10. A method comprising:
receiving baseline gene data associated with a plurality of genes for a subject, wherein the plurality of genes are sequenced from a tumor of the subject;
providing, to a predictive model, the baseline gene data; and
determining, based on the predictive model, that the subject is a candidate for a therapeutic treatment.

11. The method of claim 10, further comprising treating the subject with the therapeutic treatment.

12. The method of claim 10, further comprising training the predictive model.

13. The method of claim 12, wherein training the predictive model comprises:
determining first gene data associated with the plurality of genes;
determining second gene data associated with the plurality of genes, wherein the plurality of genes are sequenced from a plurality of tumor samples, wherein each tumor sample of the plurality of tumor samples is labeled as a responder or a non-responder;
determining, based on the first gene data and the second gene data, a plurality of features for the predictive model;
training, based on a first portion of the second gene data, the predictive model according to the plurality of features;
testing, based on a second portion of the second gene data, the predictive model; and
outputting, based on the testing, the predictive model.

14. The method of claim 13, wherein determining the first gene data associated with the plurality of genes comprises:
determining, based on the second gene data, the plurality of genes;
determining, based on the plurality of genes, one or more gene data sets that comprise at least one gene of the plurality of genes; and
generating, based on the one or more gene data sets, the first gene data.

15. The method of claim 13, wherein the first gene data is comprised of gene data from a plurality of different gene data sets.

16. The method of claim 13, wherein determining the second gene data associated with the plurality of genes comprises:
determining baseline gene expression levels for each tumor associated with the plurality of tumor samples;
treating each tumor associated with the plurality of tumor samples with a therapeutic;
determining, post-treatment, which tumors associated with the plurality of tumor samples are responders or non-responders to the therapeutic;
labeling the baseline gene expression levels for each tumor associated with the plurality of tumor samples, as responder or non-responder; and
generating, based on the labeled baseline gene expression levels, the second gene data.

17. The method of claim 15, wherein determining, based on the first gene data and the second gene data, the plurality of features for the predictive model comprises:
determining, from the first gene data, genes present in two or more of the plurality of different gene data sets as a first set of candidate genes;
determining, from the second gene data, genes of the first set of candidate genes expressed at greater than or equal to 2 Transcripts Per Million (TPM) in at least half of the plurality of tumor samples as a second set of candidate genes; and
determining, from the second gene data, genes of the second set of candidate genes with a statistically significant increase in expression level between responders and non-responders as a third set of candidate genes,
wherein the plurality of features comprises the third set of candidate genes.

18. The method of claim 17, wherein determining, based on the first gene data and the second gene data, the plurality of features for the predictive model comprises:
determining, for the third set of candidate genes, a tumor mutational burden (TMB) value for each of the plurality of tumors associated with the third set of candidate genes; and
determining, based on the TMB values, a fourth set of candidate genes,
wherein the plurality of features comprises the fourth set of candidate genes.

19. The method of claim 13, wherein training, based on the first portion of the second gene data, the predictive model according to the plurality of features results in determining a gene signature indicative of a responder.

20. The method of claim 10, wherein the therapeutic treatment is a cancer treatment.
